(19) United States
(12) Patent Application Publication
(10) Pub. No.: US 2024/0383883 A1
(43) Pub. Date: Nov. 21, 2024

(54) A PROCESS FOR PREPARATION OF ISOXAZOLE COMPOUND

(71) Applicant: UPL LIMITED, Mumbai, Maharashtra (IN)

(72) Inventors: Prashant Vasant KINI, Mumbai (IN); Sopan Nagnath GANDHALE, Mumbai (IN); Shrikant Mugutrao ANPAT, Mumbai (IN)

(21) Appl. No.: 18/684,285

(22) PCT Filed: Sep. 23, 2022
(86) PCT No.: PCT/IN2022/050849

§ 371 Date: Feb. 16, 2024

(30) Foreign Application Priority Data
Sep. 24, 2021 (IN) 202121043421

(51) Int. Cl. C07D 413/12; C07D 261/04

(52) U.S. Cl. CPC C07D 413/12; C07D 261/04

(57) ABSTRACT

The present invention relates to a novel process for preparation of an isoxazole compound of formula (I) by reacting a compound of formula (II) with a compound of formula (III). wherein, n is 0, 1 or 2; 10 R¹, R², R³ and R⁴ are independently hydrogen. C₁₋₆ alkyl, C₂₋₆ alkenyl or C₂₋₆ alkynyl; and R is linear or branched C₁₋₆ alkyl group

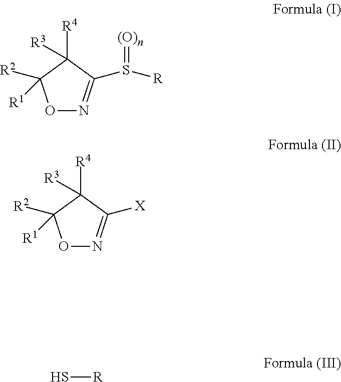

### FIELD OF THE INVENTION

The present invention relates to a process for preparation of an isoxazole compound of formula (I) which is a useful intermediate for the preparation of oxazole herbicides.

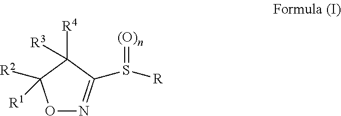

wherein, n is 0, 1 or 2;

R¹, R², R³ and R⁴ are independently hydrogen, C₁₋₆ alkyl, C₂₋₆ alkenyl or C₂₋₆ alkynyl; and R is linear or branched C₁₋₆ alkyl group

### BACKGROUND OF THE INVENTION

Isoxazole compound of formula (I) is a useful intermediate for the preparation of oxazole herbicides such as Pyroxasulfone, Fenoxasulfone and the like.

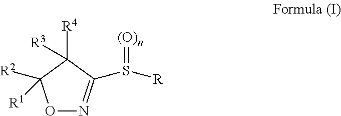

wherein, n is 0, 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group.

U.S. Pat. No. 6,841,519 discloses a process for preparation of 3-methylsulfonyl-5,5-dimethyl-2-isoxazoline wherein 3-methylthio-5,5-dimethyl-2-isoxazoline is obtained by reacting 3-chloro-5,5-dimethyl-2-isoxazoline with aqueous solution of sodium methanethiolate in presence of dimethylformamide at room temperature for 12 hours. The 3-methylthio-5,5-dimethyl-2-isoxazoline is then subjected to reaction with m-chloroperbenzoic acid at room temperature for 12-13 hours to yield 59% of 3-methylsulfonyl-5,5-dimethyl-2-isoxazoline.

Process provided in said patent is subject to industrial concerns such as low yield, long reaction time, high effluent waste and generation of dimer impurity like compound of formula (a) and compound of formula (b)

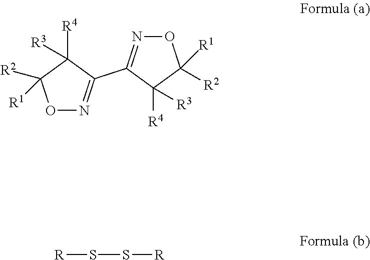

wherein, R¹, R², R³ and R⁴ are independently hydrogen, C₁₋₆ alkyl, C₂₋₆ alkenyl or C₂₋₆ alkynyl; and R is linear or branched C₁₋₆ alkyl group.

These impurities of formula (a) and formula (b) when formed or when their formation is not controlled during the process, are difficult to remove from final product and hence leads to yield and quality depletion.

U.S. Pat. No. 7,238,689 discloses a process for preparation of 5,5-dimethyl-3-ethylthio-2-isoxazoline wherein a solution containing 3-chloro-5,5-dimethyl-2-isoxazoline is reacted with ethyl mercaptan in presence of sodium hydroxide at 60 to 70° C. for 16 hours. Unfortunately, the solvent used for preparing solution containing 3-chloro-5,5-dimethyl-2-isoxazoline is not disclosed in said patent.

Hence, there remains a need to provide a process for the preparation of isoxazole compound of formula (I) which explicitly describes the process along with the reagents and solvents used and overcomes all the above discussed drawbacks of conventionally known processes and provide a process wherein the overall yield of desired product is improved using commercially available and easily handled reagents.

### OBJECT OF THE INVENTION

It is an object of the present invention to provide a process for the preparation of a compound of formula (I).

It is an object of the present invention to provide a process for the preparation of a compound of formula (I) or its salt with improved yield and purity.

Another object of the present invention is to provide a simple, cost-effective and industrially viable process for the preparation of a compound of formula (I).

### SUMMARY OF THE INVENTION

According to an aspect of the present invention, there is provided a process for preparation of an isoxazole compound of formula (I),

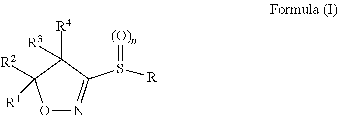

wherein, n is 0, 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group comprising,

reacting a compound of formula (II) with a compound of formula (III) in presence of triflic acid

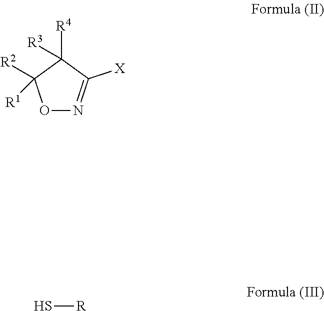

wherein, X is halogen; R¹, R², R³, R⁴ and R has same meanings as defined above.

According to another aspect of present invention, there is provided a process for preparation of an isoxazole compound of formula (I),

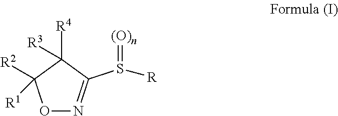

wherein, n is 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group comprising,
i) reacting a compound of formula (II) with a compound of formula (III) in presence of triflic acid to obtain a compound of formula (IV)

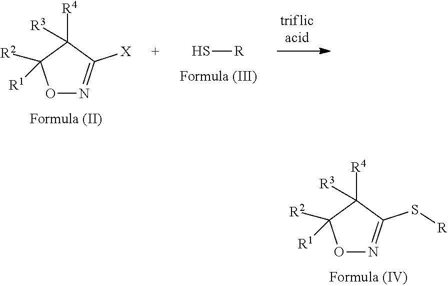

wherein, X is halogen; R¹, R², R³, R⁴ and R has same meanings as defined above
ii) oxidizing the compound of formula (IV) to obtain an isoxazole compound of formula (I)

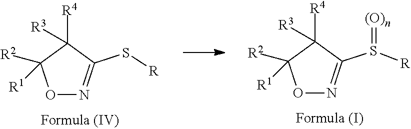

According to another aspect of present invention, there is provided a process for preparation of an isoxazole compound of formula (I),

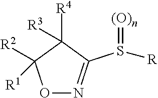

wherein, n is 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group

comprising, oxidizing a compound of formula (IV) to obtain an isoxazole compound of formula (I)

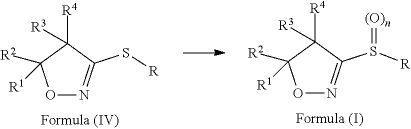

wherein the oxidation is carried out in presence of an oxidizing agent such as oxone or hydrogen peroxide.

According to yet another aspect of present invention, there is provided a process for preparation of Pyroxasulfone of formula (V)

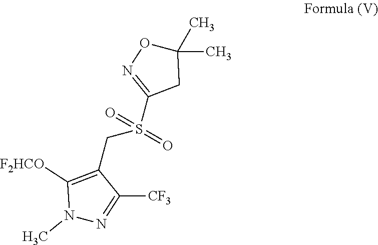 [SMILES: Cn1nc(C(F)(F)F)c(CS(=O)(=O)C2=NOC(C)(C)C2)c1OC(F)F]

using an isoxazole compound of formula (I),

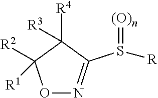

wherein, n is 0, 1 or 2;

R¹, R², R³ and R⁴ are independently hydrogen, C₁₋₆ alkyl, C₂₋₆ alkenyl or C₂₋₆ alkynyl;

R is linear or branched C₁₋₆ alkyl group

and wherein the isoxazole compound of formula (I) is prepared by reacting a compound of formula (II) with a compound of formula (III) in the presence of triflic acid

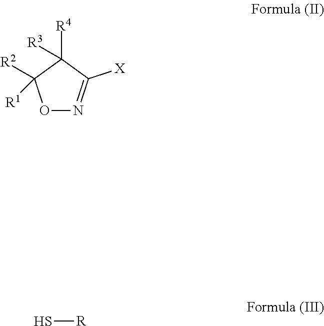

wherein, X is halogen; R¹, R², R³, R⁴ and R has same meanings as defined above.

According to yet another aspect of present invention, there is provided a process for preparation of Pyroxasulfone of formula (V)

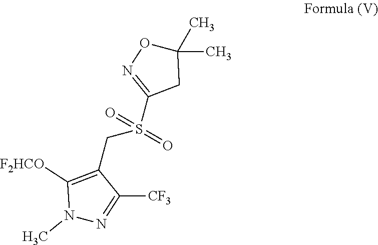 [SMILES: Cn1nc(C(F)(F)F)c(CS(=O)(=O)C2=NOC(C)(C)C2)c1OC(F)F]

using an isoxazole compound of formula (I),

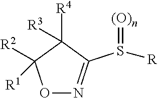

wherein, n is 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group

and wherein the isoxazole compound of formula (I) is prepared by oxidizing a compound of formula (IV)

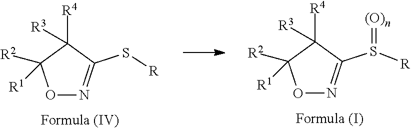

wherein the oxidation is carried out in presence of an oxidizing agent such as oxone or hydrogen peroxide.

### DETAILED DESCRIPTION OF THE INVENTION

### Definitions

For convenience, before further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or ±5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The term “room temperature” unless stated otherwise, essentially means temperature in range of 15 to 45° C.

As used herein in this context, the expression “substantially free” will be understood to mean that isoxazole compound of formula (I) contains 20% or less, 10% or less, 5% or less, 2% or less, or 1% or less or 0.5% or less of any known or unknown impurity as measured, for example, by HPLC.

The term “purity” means purity as determined by HPLC (“High Pressure Liquid Chromatography”).

The term “about” shall be interpreted to mean “approximately” or “reasonably close to” and any statistically insignificant variations therefrom.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

As used herein, the term “(C₁-C₆) alkyl” or “alkyl” alone or as part of a substituent group, refers to the radical of saturated aliphatic groups, including straight or branched-chain alkyl groups. A straight-chain or branched chain alkyl has six or fewer carbon atoms in its backbone, for instance, C₁-C₆ for straight chain and C₃-C₆ for branched chain. As used herein, (C₁-C₆) alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

As used herein, the term “C₂₋₆ alkenyl” or “alkenyl” alone or as part of a substituent group, refers to a straight or branched chain hydrocarbon containing 2 to 6 carbon atoms, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

As used herein, the term “C2-6 alkynyl” or “alkynyl” alone or as part of a substituent group, refers to the radical of a straight or branched chain hydrocarbon group containing from 2 to 6 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “halogen” refers to chlorine, fluorine, bromine, or iodine atom.

The present disclosure is not to be limited in scope by the specific embodiments described herein, which are intended for the purposes of exemplification only.

According to an aspect of the present invention, there is provided a process for preparation of an isoxazole compound of formula (I),

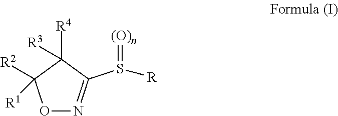

wherein, n is 0, 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group

comprising,

reacting a compound of formula (II) with a compound of formula (III) in presence of triflic acid

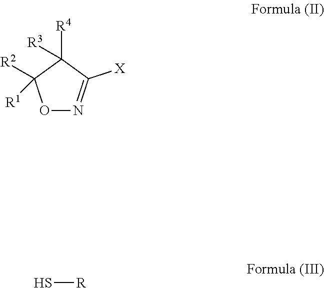

wherein, X is halogen; R¹, R², R³, R⁴ and R has same meanings as defined above.

In an embodiment, in the compound of formula (I), R¹, R² are C₁₋₆ alkyl; R³, R⁴ are independently hydrogen; and R is a linear C₁₋₆ alkyl group.

In an embodiment, in the compound of formula (I), n is 0, R¹, R² are C₁₋₆ alkyl; R³, R⁴ are independently hydrogen; and R is a linear C₁₋₆ alkyl group.

In an embodiment, in the compound of formula (I), R¹, R² are methyl or ethyl group independently; R³, R⁴ are independently hydrogen; and R is methyl or ethyl group.

In an embodiment, the process provides the compound of formula (I) wherein n is 0; R¹, R², R³, R⁴ and R has same meanings as defined above; and is represented as compound of formula (IV)

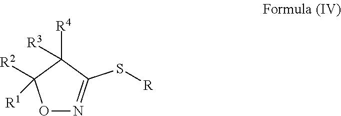

In an embodiment, in the compound of formula (II), R¹, R² are C₁₋₆ alkyl; R³, R⁴ are independently hydrogen; and X is halogen.

In an embodiment, in compound of formula (II), R¹, R² are methyl group, R³, R⁴ are independently hydrogen and X is bromine.

In an embodiment, in compound of formula (III), R is a linear C₁₋₆ alkyl group.

In an embodiment, in compound of formula (III), R is ethyl group.

In an embodiment, the amount of triflic acid used may vary from catalytic amount to 0.5 moles with respect to compound of formula (II).

In an embodiment, the amount of triflic acid used may vary from 0.001 to 0.5 moles with respect to compound of formula (II).

In an embodiment, the reaction of compound of formula (II) with compound of formula (III) is carried out in presence of suitable solvent.

The suitable solvent used is selected from, but not limited to, chlorinated solvent, ether, ester, amide, alkyl sulfoxide, alkyl sulfone, or mixtures thereof.

The suitable solvent used is selected from, but not limited to, chlorinated solvents such as dichloromethane, dichloroethane, chloroform, 1,1,2-trichloroethane, chlorobenzene, o-dichlorobenzene and the like; ethers such as tetrahydrofuran, dioxane and the like; esters like ethyl acetate, n-butyl acetate and the like; amides such as dimethylformamide and the like; alkyl sulfoxides or sulfones such as dimethyl sulfoxide, dimethyl sulfone and the like; or mixtures thereof.

In an embodiment, the reaction of compound of formula (II) with compound of formula (III) is carried out in presence of dichloromethane as solvent.

In an embodiment, the reaction of compound of formula (II) with compound of formula (III) is carried out at temperature ranging from 0° C. to 150° C., preferably from 0° C. to 100° C.

In an embodiment, the process for preparation of isoxazole compound of formula (I) proceeds through formation of compound of formula (IV)

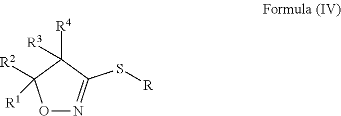

wherein R¹, R², R³, R⁴ and R has same meanings as defined above.

The compound of formula (IV) may or may not be isolated.

In an embodiment, the process for preparation of isoxazole compound of formula (I) comprises oxidising compound of formula (IV) with an oxidising agent to obtain desired compound of formula (I).

In an embodiment, the oxidising agent used is selected from, but not limited to, hydrogen peroxide, oxone, m-chloroperbenzoic acid, performic acid, peracetic acid, potassium permanganate, sodium periodate and the like.

In an embodiment, the oxidising agent used is oxone.

In an embodiment, the oxidising agent used is hydrogen peroxide.

In an embodiment, the oxidation of compound of formula (IV) is carried out in presence of a metal catalyst.

The catalyst used can be a metal catalyst such as tungsten catalyst, molybdenum catalyst, titanium catalyst, zirconium catalyst or mixture thereof

The tungsten catalyst used may be selected from tungsten, tungstic acid, tungstic acid salt, metallic tungsten, tungsten oxide, tungsten carbide o mixtures thereof.

The tungsten catalyst such as tungsten chloride, tungsten bromide, tungsten sulfide, phospho tungstic acid or a salt thereof, tungstic acid or a salt thereof, sodium tungstate, potassium tungstate, calcium tungstate, lithium tungstate, tungsten tungstate, coordination complex of tungsten or mixture thereof, may be used.

Preferably, the tungsten catalyst used is sodium tungstate, more preferably sodium tungstate dihydrate is used.

The molybdenum catalyst used may be selected from molybdic acid, molybdates, metallic molybdenum, molybdenum carbide, molybdenum oxide, molybdenum chloride or mixtures thereof.

The molybdenum catalyst such as molybdate, sodium molybdate, potassium molybdate, ammonium molybdate, molybdate oxide (VI), molybdenum carbide, molybdenum chloride (V), molybdate sulfide (IV), phosphomolybdate, sodium phosphomolybdate, ammonium phosphomolybdate, silicate molybdate or mixtures thereof, coordination complex of molybdenum may be used.

Preferably, the molybdenum catalyst used is ammonium molybdate, more preferably ammonium molybdate tetrahydrate is used.

The titanium catalyst used may be selected from titanic acid, titanate, titanium oxide, titanium carbide, titanium chloride and mixtures thereof.

The zirconium catalyst used may be selected from zirconium oxide, zirconium carbide, zirconium chloride and mixtures thereof.

In an embodiment, the amount of metal catalyst used may vary from catalytic amount to 0.25 moles with respect to compound of formula (II).

In an embodiment, the oxidation of compound of formula (IV) is carried out in presence of solvent selected from but not limited to, group comprising of an organic acid such as acetic acid, ethers such as tetrahydrofuran, dioxane and the like; nitriles such as acetonitrile; alcohols such as methanol, ethanol, isopropanol and the likes; water or mixture thereof.

In an embodiment, the oxidation of compound of formula (IV) is carried out in presence of tetrahydrofuran as solvent.

In an embodiment, the oxidation of compound of formula (IV) is carried out at temperature ranging from 0 to 100° C.

In an embodiment, the process of present invention provides an isoxazole compound of formula (I) wherein n is 2, substantially free of monoxide impurity.

In an embodiment, the isoxazole compound of formula (I) wherein n is 2, substantially free of monoxide impurity, can be prepared in high yield and purity.

According to another aspect of present invention, there is provided a process for preparation of an isoxazole compound of formula (I),

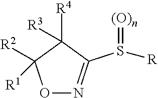

wherein, n is 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group

comprising,
i) reacting a compound of formula (II) with a compound of formula (III) in presence of triflic acid to obtain a compound of formula (IV)

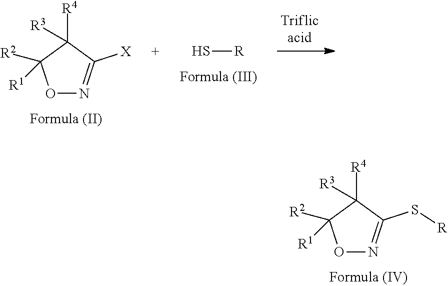

wherein, X is halogen; R¹, R², R³, R⁴ and R has same meanings as defined above
ii) oxidizing the compound of formula (IV) to obtain an isoxazole compound of formula (I)

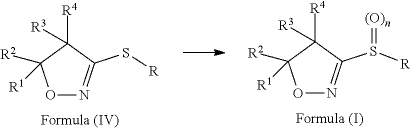

In an embodiment, there is provided a process for preparation of an isoxazole compound of formula (IV),

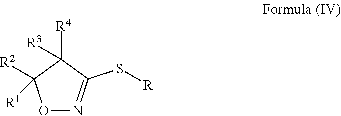

wherein, R¹, R², R³ and R⁴ are independently hydrogen, C₁₋₆ alkyl, C₂₋₆ alkenyl or C₂₋₆ alkynyl; and

R is linear or branched C₁₋₆ alkyl group

comprising, reacting a compound of formula (II) with a compound of formula (III) in presence of triflic acid to obtain a compound of formula (IV)

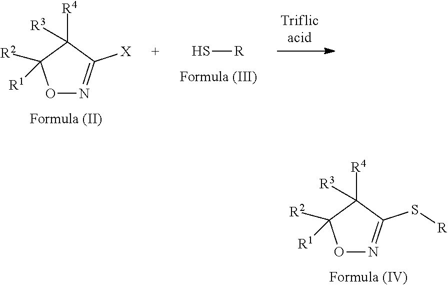

wherein, X is halogen; R¹, R², R³, R⁴ and R has same meanings as defined above

In an embodiment, the step ii) oxidation is carried out in presence of an oxidising agent.

In an embodiment, the oxidising agent is selected from the group comprising of hydrogen peroxide, oxone, m-chloroperbenzoic acid, performic acid, peracetic acid, potassium permanganate or sodium periodate.

In an embodiment, the amount of oxidising agent used may vary from the range of 1 to 8 moles with respect to compound of formula (II).

In an embodiment, the step ii) oxidation is carried out in presence of a metal catalyst.

In an embodiment, the metal catalyst is selected from the group comprising of tungsten catalyst, molybdenum catalyst, titanium catalyst, zirconium catalyst or mixture thereof.

The tungsten catalyst used may be selected from tungsten, tungstic acid, tungstic acid salt, metallic tungsten, tungsten oxide, tungsten carbide o mixtures thereof.

The tungsten catalyst such as tungsten chloride, tungsten bromide, tungsten sulfide, phospho tungstic acid or a salt thereof, tungstic acid or a salt thereof, sodium tungstate, potassium tungstate, calcium tungstate, lithium tungstate, tungsten tungstate, coordination complex of tungsten or mixture thereof, may be used.

Preferably, the tungsten catalyst used is sodium tungstate, more preferably sodium tungstate dihydrate is used.

The molybdenum catalyst used may be selected from molybdic acid, molybdates, metallic molybdenum, molybdenum carbide, molybdenum oxide, molybdenum chloride or mixtures thereof.

The molybdenum catalyst such as molybdate, sodium molybdate, potassium molybdate, ammonium molybdate, molybdate oxide (VI), molybdenum carbide, molybdenum chloride (V), molybdate sulfide (IV), phosphomolybdate, sodium phosphomolybdate, ammonium phosphomolybdate, silicate molybdate or mixtures thereof, coordination complex of molybdenum may be used.

Preferably, the molybdenum catalyst used is ammonium molybdate, more preferably ammonium molybdate tetrahydrate is used.

The titanium catalyst used may be selected from titanic acid, titanate, titanium oxide, titanium carbide, titanium chloride and mixtures thereof.

The zirconium catalyst used may be selected from zirconium oxide, zirconium carbide, zirconium chloride and mixtures thereof.

In an embodiment, the amount of metal catalyst used may vary from catalytic amount to 0.25 moles with respect to compound of formula (II).

In an embodiment, the step ii) oxidation is carried out in presence of a solvent.

The solvent used is selected the group comprising of an organic acid, ether, nitrile, alcohol water or mixture thereof.

The solvent used is selected from but not limited to, an organic acid such as acetic acid, ethers such as tetrahydrofuran, dioxane and the like; nitriles such as acetonitrile; alcohols such as methanol, ethanol, isopropanol and the likes; water or mixture thereof.

In an embodiment, the oxidation of compound of formula (IV) is carried out in presence of tetrahydrofuran as solvent.

In an embodiment, the step ii) oxidation is carried out at temperature ranging from 0 to 100° C.

In an embodiment, step i) and step ii) are carried out in one pot process.

In an embodiment, the isoxazole compound of formula (I) wherein R¹, R² are methyl groups independently; R³, R⁴ are independently hydrogen; and R is ethyl group; is prepared according to process of the present invention and is represented by scheme 1

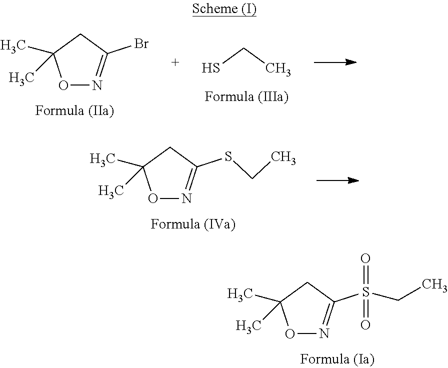

According to another aspect of present invention, there is provided a process for preparation of an isoxazole compound of formula (I),

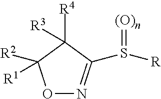

wherein, n is 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group

comprising, oxidizing a compound of formula (IV) to obtain an isoxazole compound of formula (I)

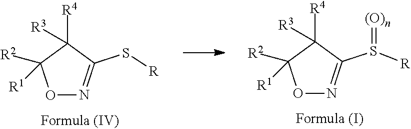

wherein the oxidation is carried out in presence of an oxidizing agent.

In an embodiment, the oxidising agent is selected from, but not limited to, oxone or hydrogen peroxide.

In an embodiment, there is provided a process for preparation an isoxazole compound of formula (I),

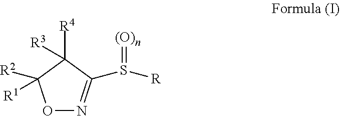

wherein, n is 0, 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group

comprising, oxidizing a compound of formula (IV) to obtain an isoxazole compound of formula (I)

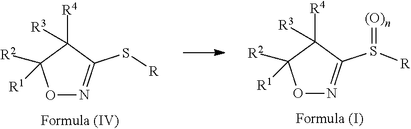

wherein the oxidation is carried out in presence of oxone.

In an embodiment, there is provided a process for preparation an isoxazole compound of formula (I),

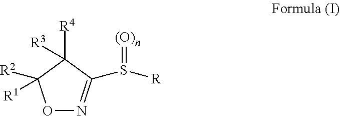

wherein, n is 0, 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group

comprising, oxidizing a compound of formula (IV) to obtain an isoxazole compound of formula (I)

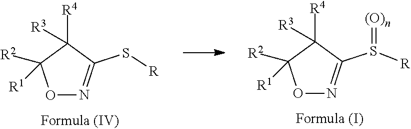

wherein the oxidation is carried out in presence of hydrogen peroxide and a metal catalyst.

According to yet another aspect of present invention, there is provided a process for preparation of Pyroxasulfone of formula (V)

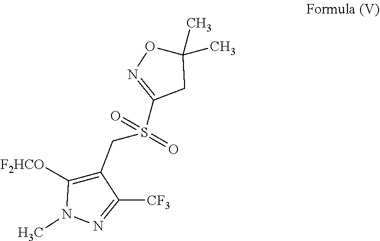

using an isoxazole compound of formula (I),

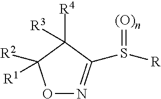

wherein, n is 0, 1 or 2;

R¹, R², R³ and R⁴ are independently hydrogen, C₁₋₆ alkyl, C₂₋₆ alkenyl or C₂₋₆ alkynyl;

R is linear or branched C₁₋₆ alkyl group

and wherein the isoxazole compound of formula (I) is prepared by reacting a compound of formula (II) with a compound of formula (III) in the presence of triflic acid

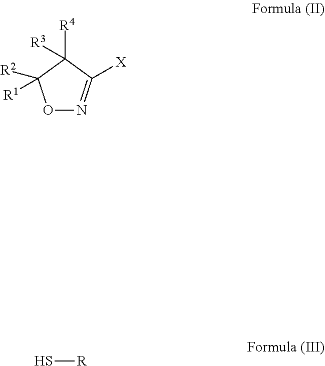

wherein, X is halogen; R¹, R², R³, R⁴ and R has same meanings as defined above.

According to yet another aspect of present invention, there is provided a process for preparation of Pyroxasulfone of formula (V)

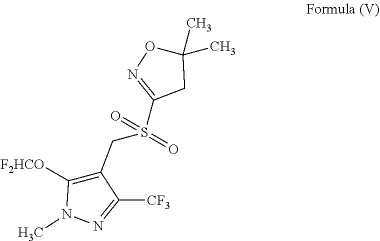

using an isoxazole compound of formula (I),

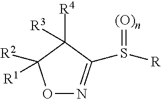

wherein, n is 1 or 2;

R¹, R², R³ and R⁴ are independently hydrogen, C₁₋₆ alkyl, C₂₋₆ alkenyl or C₂₋₆ alkynyl; and

R is linear or branched C₁₋₆ alkyl group

and wherein the isoxazole compound of formula (I) is prepared by oxidizing a compound of formula (IV)

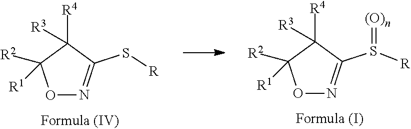

wherein the oxidation is carried out in presence of an oxidizing agent such as oxone or hydrogen peroxide.

Various process for preparation of Pyroxasulfone are provided in prior art proceeding through the isoxazole compound of formula (I) any of such prior art process can be followed to obtain Pyroxasulfone of formula (V).

According to the processes known in the prior art, the isoxazole compound of formula (I) wherein n is 0, can be obtained by reacting compound of formula (II) with sodium alkylthiolates in presence of solvent such as dimethylformamide at temperature ranging from room temperature to 70° C. The isoxazole compound of formula (I) wherein n is 0, obtained can be further oxidized to the get isoxazole compound of formula (I) wherein n is 1 or 2. The process known in prior art utilises the oxidizing agent such as m-chloroperbenzoic acid for such reactions. However, the yield obtained by known process is not satisfactory. Secondly, in the work-up procedure of the prior art process the isoxazole compound of formula (I) wherein n is 0; for isolation, requires use of an addition solvent such as ethyl acetate, thereby increasing the industrial effluents and their treatment expenditures. Lastly, use of oxidizing agent such as m-chloroperbenzoic acid to obtain the isoxazole compound of formula (I) wherein n is 1 or 2, is not feasible on industrial scale due to its scarcity in good quality in market.

Above discussed prior art processes are known in the literature and described, for example, in U.S. Pat. Nos. 6,841,519 and 7,238,689.

In another embodiment there is provided use of Pyroxasulfone in the preparation of agrochemical composition or formulation.

In another embodiment there is provided use of Pyroxasulfone prepared using the process disclosed in the present invention in the preparation of agrochemical composition or formulation.

According to another embodiment, the present invention provides a herbicidal composition comprising pyroxasulfone prepared according to the process as described herein and at least one agrochemically acceptable excipient.

According to another embodiment, the present herbicide composition further comprising at least one additional herbicide.

In an embodiment, the additional herbicide is selected from 2,4-D, anisiflupurin, acetochlor, aclonifen, amicarbazone, halauxifen, halauxifen-methyl, florpyrauxifen, amidosulfuron, aminocyclopyrachlor, aminopyralid, aminotriazole, ammonium thiocyanate, anilofos, asulam, azimsulfnron, atrazine, beflubutamid, benazolin, benfuresate, bensulfuron-methyl, bentazon-sodium, benzofenap, bifenox, bixlozone, benquitrione, bipyrazone, bispyribacsodium, bromobutide, bromacil, bromoxynil, butachlor, butafenacil, butralin, butroxydim, carbetamide, cafenstrole, carfentrazone, carfentrazone-ethyl, chlormequat, clopyralid, chlorsulfuron, chlortoluron, cinidon-ethyl, imazethapyr, clodinafop-propargyl, clomeprop, clomazone, cloransulam-methyl, cyanazine, cyclopyrimorate, cyclosulfamuron, cycloxydim, cyhalofop-butyl, cypyrafluone, daimuron, dicamba, dichlobenil, dichlorprop-P, diclofop-methyl, diclosulam, diflufenican, diflufenzopyr, dimefuron, dimethachlor, diquat, dioxopyritrione, diuron, 5-ethyl dipropylcarbamothioate (EPTC), esprocarb, ethoxysulfuron, etobenzanid, fenoxaprop, fenoxaprop-ethyl, fenoxapropethyl+isoxadifen-ethyl, fenoxaprop-P-ethyl, fenoxasulfone, fenquinotrione, fentrazamide, fenpyrazone, flazasulfuron, florasulam, fluazifop, fluazifop-P-butyl, flucarbazone, flucarbazone-sodium, flucetosulfuron, flufenacet, flumetsulam, flumioxazin, flupyrsulfuron, flurochloridone, fluroxypyr. fluroxypyr-meptyl, flurtamone, glufosinate, glufosinate-ammonium, glyphosate, halosulfuron-methyl, haloxyfop-methyl, haloxyfop-R-methyl, hexazinone, imazamethabenz, imazamox, imazapic, imazapyr, imazaquin, imazosulfuron, indanofan, indaziflam, iodosulfuron, iodosulfuron-ethyl-sodium, iofensulfuron, ioxynil, ipfencarbazone, isoproturon, isoxaben, isoxaflutole, lactofen, linuron, MCPA, MCPB, mecoprop-P, mefenacet, mesosulfuron, mesosulfuron-ethyl sodium, mesotrione, metamifop, metazochlor, metazosulfuron, metosulam, metribuzin, metsulfuron, metsulfuronmethyl, molinate, MSMA, napropamide, napropamide-M, orfurazon, orthosulfamuron, oryzalin, oxadiargyl, oxadiazon, oxazichlomefone, oxyfluorfen, paraquat, pendimethalin, penoxsulam, pentoxazone, pethoxamid, picloram, picolinafen, pinoxaden, pretilachlor, primisulfuron, profluazol, profoxydim, propanil, propaquizafop, propyrisulfuron, propoxycarbazone, propyzamide, prosulfocarb, prosulfuron, pyraclonil, pyraflufen-ethyl, pyrasulfotole, pyrazosulfuron-ethyl, pyrazolynate, pyribenzoxim, L-glufosinate or its salts, pyributicarb, pyridate, pyriftalid, pyrimisulfan, pyroxsulam, pyroxasulfone, quinclorac, quinmerac, quizalofop-ethyl-D, quizalofop-P-ethyl, quizalofop-P-tefuryl, rimsulfuron, rimisoxafen, sethoxydim, simazine, sulcotrione, sulfentrazone, sulfometuron, sulfosate, sulfosulfuron, tebuthiuron, tefuryltrione, tepraloxidim, terbacil, terbuthylazine, terbutryn, tetflupyrolimet, thenylchlor, thiazopyr, thifensulfuron, thifensulfuron-methyl, thiobencarb, topramezone, tralkoxydim, triafamone, triasulfuron, tribenuron, tribenuron-methyl, triafamone, triclopyr, tripyrasulfone, SL-1201, cyclopyranil, dimesulfazet, epyrifenacil, tembotrione, thiencarbazone methyl, flucetosulfuron, pyrasulfotole, saflufenacil, pyraclonil, fenquinotrione, tiafenacil, cinmethylin, lancotrione-sodium, trifludimoxazin, cyclopyrimorate, methiozolin, aminocyclopyrachlor, bicyclopyrone, triafamone, tolpyralate. trifluralin, and agriculturally acceptable salts, choline salts, esters and mixtures thereof.

The process provided in the present invention, for preparation of the isoxazole compound of formula (I) has proven benefits over all the prior art processes. Some of them are listed as below:

### Advantages of the Present Invention

1. The process provides desired isoxazole compound of formula (I) in high yield.
2. The volumes of industrial effluents generated in preparation of the isoxazole compound of formula (I) are reduced.
3. The formation dimer impurities are controlled to great extent in the process, thereby improving quality of final product.
4. Utilizes easily available oxidizing agents which ensures reaction completion.
5. Provides a simple, cost-effective and industrially viable process for preparation of the isoxazole compound of formula (I).

The process for preparation of the isoxazole compound of formula (I) according to the invention is ascertained by the experiments as exemplified below. These examples are merely illustrations and are not to be understood as limiting the scope and underlying principles of the invention in any way. Indeed, various modifications of the invention in addition to those shown and described herein will become apparent to those skilled in the art from the following examples and foregoing description.

### EXAMPLES

The following examples are presented to provide what is believed to be the most useful and readily understood description of procedures and conceptual aspects of this invention. The examples provided below are merely illustrative of the invention and are not intended to limit the same to disclosed embodiments. Variations and changes obvious to one skilled in the art are intended to be within the scope and nature of the invention.

### Analytical Method Details

Samples were analysed on high performance liquid chromatograph with UV detector and integrator/software at 220 nm using Zorbax SB C-8 (250 mm×4.6 mm, 5μ).

### Example 1

Synthesis of 3-(ethylsulfonyl)-5,5-dimethyl-4,5-dihydro-1,2-oxazole (Compound of Formula Ia)
Step 1: 3-(ethylsulfanyl)-5,5-dimethyl-4,5-dihydro-1,2-oxazole (Compound of Formula IVa)

100 g of 3-bromo-5,5-dimethyl-4,5-dihydro-1,2-oxazole was added in 750 ml of dichloromethane to form a solution. The solution was cooled at a temperature in the range from 0 to 5° C. To said cooled solution 21.07 g of triflic acid was added with continuous stirring. Further, 61 g of ethyl mercaptan was added to this mixture and after addition the temperature was raised to a temperature in the range from 15 to 20° C. The reaction mixture was maintained at 5 to 20° C. for 12 to 14 hours. After completion of reaction, 300 g of water was added to the reaction mixture, the mixture was stirred for 15 to 30 mins and the layers were separated. The organic layer was washed with water and 20% sodium chloride solution. The organic layer was then concentrated under vacuum at 40° C. and degassed to obtain 100 g of 3-(ethylsulfanyl)-5,5-dimethyl-4,5-dihydro-1,2-oxazole (Yield: 95.18%).

Step 2: Oxidation of 3-(ethylsulfanyl)-5,5-dimethyl-4,5-dihydro-1,2-oxazole to 3-(ethylsulfonyl)-5,5-dimethyl-4,5-dihydro-1,2-oxazole (Compound of Formula Ia)

To a mixture containing 95.1 g of 3-(ethylsulfanyl)-5,5-dimethyl-4,5-dihydro-1,2-oxazole and 450 g of tetrahydrofuran (THF), solution of 6.70 g of sodium tungstate dihydrate in 67 g of water was added. The mixture was then heated to a temperature in the range from 50 to 55° C. and 169.7 g of 50% hydrogen peroxide was slowly added in 1 to 2 hour. Thereafter, the reaction was maintained at 60 to 65° C. for 2 to 3 hours. After completion of reaction, the reaction mass was concentrated and to the concentrated mass 250 g of water was added. The mixture was cooled to a temperature in the range from 0 to 5° C. and stirred for 0.5 to 1 hour at the said temperature to precipitate out the product. The product thus, obtained was filtered, washed with water and dried to obtain 99.10 g of 3-(ethylsulfonyl)-5,5-dimethyl-4,5-dihydro-1,2-oxazole (melting point: 59-62° C.). (HPLC Purity: 98.31%; Yield: 79.34%).

1. A process for preparation of an isoxazole compound of formula (I), 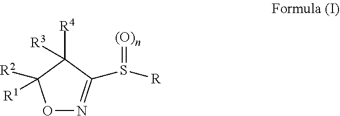
wherein, n is 0, 1 or 2; R¹, R², R³ and R⁴ are independently hydrogen, C1-6 alkyl, C2-6 alkenyl or C₂₋₆ alkynyl; and R is linear or branched C₁₋₆ alkyl group, the process comprising,
reacting a compound of formula (II) with a compound of formula (III) in presence of triflic acid; 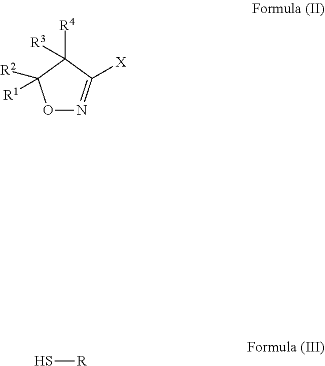
wherein, X is halogen; R¹, R², R³, R⁴ and R has same meanings as defined above.

2. The process as claimed in claim 1, wherein in the compound of formula (I), R¹, R² are independently C₁₋₆ alkyl; R³, R⁴ are independently hydrogen; and R is linear C₁₋₆ alkyl group.

3. The process as claimed in claim 1, wherein n is 0; R¹, R² are independently C₁₋₆ alkyl;
R³, R⁴ are independently hydrogen; and R is linear C₁₋₆ alkyl group in the compound of formula I.

4. The process as claimed in claim 3, wherein the compound of formula I is represented as compound of formula (IV).

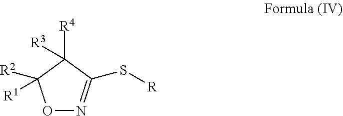

5. The process as claimed in claim 1, wherein said reaction is carried out in presence of a solvent.

6. The process as claimed in claim 5, wherein said solvent is selected from the group comprising of chlorinated solvent, ether, ester, amide, alkyl sulfoxide, alkyl sulfone, or mixtures thereof.

7. A process for preparation of an isoxazole compound of formula (I), 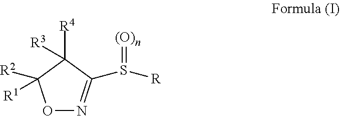
wherein, n is 1 or 2;
R¹, R², R³ and R⁴ are independently hydrogen, C₁₋₆ alkyl, C₂₋₆ alkenyl or C₂₋₆ alkynyl; and
R is linear or branched C₁₋₆ alkyl group
comprising,
i) reacting a compound of formula (II) with a compound of formula (III) in presence of triflic acid to obtain a compound of formula (IV) 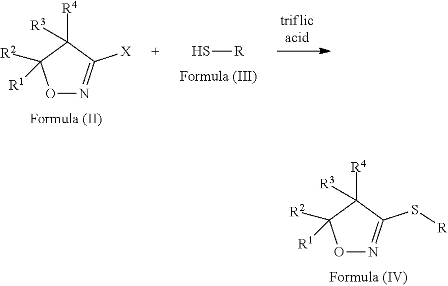
wherein, X is a halogen; R¹, R², R³ and R⁴ are independently hydrogen, C₁₋₆ alkyl, C₂₋₆ alkenyl or C₂₋₆ alkynyl and R is linear or branched C₁₋₆ alkyl group; and
ii) oxidizing the compound of formula (IV) to obtain an isoxazole compound of formula (I)

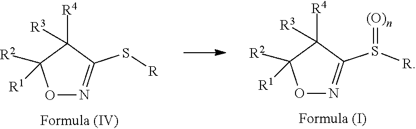

8. The process as claimed in claim 7, wherein said oxidation is carried out in presence of an oxidising agent.

9. The process as claimed in claim 8, wherein said oxidising agent is selected from the group comprising of hydrogen peroxide, oxone, m-chloroperbenzoic acid, performic acid, peracetic acid, potassium permanganate or sodium periodate.

10. The process as claimed in claim 7, wherein said oxidation is carried out in presence of a metal catalyst.

11. The process as claimed in claim 10, wherein said metal catalyst is selected from the group comprising of tungsten catalyst, molybdenum catalyst, titanium catalyst, zirconium catalyst or mixture thereof.

12. The process as claimed in claim 7, wherein said oxidation is carried out in presence of a solvent.

13. The process as claimed in claim 12, wherein said solvent is selected the group comprising of an organic acid, ether, nitrile, alcohol water or mixture thereof.

14. A process for preparation of pyroxasulfone of formula (V) 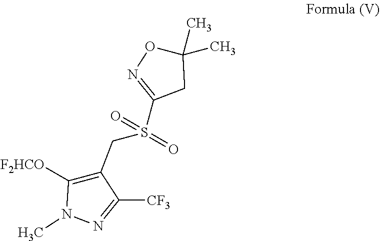
using an isoxazole compound of formula (I), 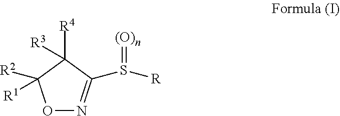
wherein, n is 0, 1 or 2; R¹, R², R³ and R⁴ are independently hydrogen, C₁₋₆ alkyl, C₂₋₆ alkenyl or C₂₋₆ alkynyl; R is linear or branched C₁₋₆ alkyl group; and
wherein the isoxazole compound of formula (I) is prepared by process as claimed in claim 1 or 5.

15. A process for preparation of an isoxazole compound of formula (IV), 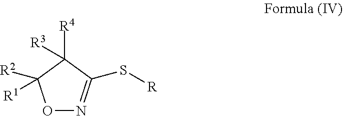
wherein, R¹, R², R³ and R⁴ are independently hydrogen, C₁₋₆ alkyl, C₂₋₆ alkenyl or C₂₋₆ alkynyl; and R is linear or branched C₁₋₆ alkyl group, the process comprising, reacting a compound of formula (II) with a compound of formula (III) in presence of triflic acid to obtain a compound of formula (IV) 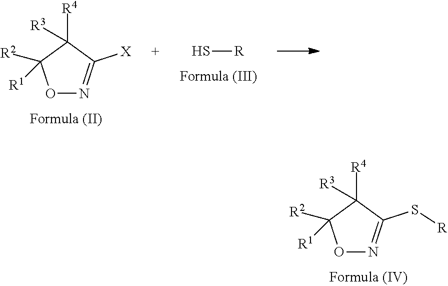
wherein, X is halogen; R¹, R², R³, R⁴ and R has same meanings as defined above.
